(19) United States
(12) Patent Application Publication
(10) Pub. No.: US 2023/0227767 A1
(43) Pub. Date: Jul. 20, 2023

(54) BIOPROCESSING WITH IMPROVED MEASUREMENT OF PROCESS PARAMETERS, RELATED APPARATUS AND METHODS

(71) Applicant: UNIVERCELLS TECHNOLOGIES S.A., Nivelles (BE)

(72) Inventors: Bastien Mairesse, Uccle (BE); Jean-Christophe Drugmand, Wavre (BE)

(21) Appl. No.: 17/924,224

(22) PCT Filed: May 11, 2021
(86) PCT No.: PCT/EP2021/062536

§ 371 Date: Nov. 9, 2022

Related U.S. Application Data
(60) Provisional application No. 63/022,835, filed on May 11, 2020.

(51) Int. Cl. C12M 1/36; C12M 1/06; C12M 1/12

(52) U.S. Cl. CPC C12M 41/48; C12M 27/02; C12M 25/06

(57) ABSTRACT

A system includes a bioprocessing vessel formed of a material insulative to a liquid medium when present therein. At least one sensor is for sensing a parameter of the bioprocessing vessel and generating a signal indicative of the parameter. At least ne conductor is adapted to provide electrical communication between the liquid medium in the bioprocessing vessel and an external structure to achieve equipotential for reducing noise in the signal produced by the at least one sensor. Related apparatus and methods are also disclosed.

This application claims the benefit of U.S. Provisional Patent Application Ser. No. 63/022,835, filed May 11, 2020, the disclosure of which is incorporated herein by reference.

### TECHNICAL FIELD

This document relates generally to the bioprocessing arts and, more particularly, to a bioprocessing vessel or an associated system providing enhanced resistance to noise by achieving equipotential with an associated liquid medium, thus improving the measurement of one or more process parameters by associated sensors.

### BACKGROUND

Cell culture vessels such as bioreactors are frequently used for bioprocessing, such as for culturing cells for biologics research and manufacturing. A culture medium is added to the bioreactor to feed the cells being grown therein. A certain level of gas transfer between the gas phase and the liquid phase of the culture medium is also necessary to permit optimal cell growth.

In the course of cell culturing in particular, it is also necessary to obtain measurement of one or more process parameters in order to obtain data relating to the operating conditions of the bioreactor. One or more sensors or probes are often used for this purpose. For example, a temperature probe or sensor may be provided for sensing the temperature of the culture medium. Additional sensors, such as pH, oxygen, dissolved oxygen (DO), temperature, biomass/cell density, glucose, lactate, etc. may also be utilized for process control.

To optimize the signals such sensors provide, it is desirable for the liquid medium within the bioreactor to be at the same potential (equipotential) as the bioreactor and associated sensors or probes. The term equipotential relates to all conductive objects in a space having the same level of electrical charge or lack thereof. The lack of equipotential results in electromagnetic noise, which affects the accuracy of any sensor or probe measurements in proximity of the bioreactor.

Typically, stainless steel bioreactors do not suffer from such noise because they utilize ground connector(s) to eliminate potential differences among the liquid media, the bioreactor and the associated sensors or probes. On the other hand, as shown in FIG. 1, a disposable vessel for bioprocessing, such as a bioreactor 10 for culturing cells, is typically manufactured from insulating or non-conductive materials (e.g., plastics such as polyethylene or the like). Such a bioreactor 10 can suffer from noise as the plastic material serves to insulate the liquid media. Specifically, in such an arrangement, the sensors or probes 12 are positioned through ports in the walls or lid 14 of such bioreactor within insulated receiving bodies 16 that may extend into the interior of the bioreactor 10. This configuration suffers from a difference in potential that can create electrical interference, such as by induced current and increased electromagnetic noise, which affects measurement data collected from the associated sensor(s). In situations where a bioreactor includes a source of electrostatic energy, such as a magnetic mixer, an alternating current driven heating system, an external pump, or even a tubing connection to such a source, the resulting induced current created by the presence of electrical interference may lead to undesirable consequences in terms of signal processing and, thus, potential issues with control of the bioprocessing.

Accordingly, a need is identified for a bioprocessing arrangement, such as one involving a cell culturing vessel, bioreactor, or a system for bioprocessing, that is insulative to the liquid medium (in the case of a plastic, disposable vessel, for example), and yet adapted to reduce or eliminate signal noise caused by electrical interference in an associated sensor to achieve better measurement of process conditions.

### SUMMARY

According to one aspect of the disclosure, a system for bioprocessing comprises a bioprocessing vessel formed of a material insulative to a liquid medium when present therein. At least one sensor is provided for sensing a parameter of the bioprocessing vessel and generating a signal indicative of the parameter. At least one conductor is adapted to provide electrical communication between the liquid medium in the bioprocessing vessel and an external structure to achieve equipotential for reducing noise in the signal produced by the at least one sensor.

In one embodiment, the at least one conductor comprises a conductive material connecting the liquid medium with the external structure. For example, the at least one conductor may be a wire, cable, or pin made of a metal, such as stainless steel or copper.

The sensor may comprise a body connected (directly or indirectly) to the insulative material of the bioprocessing vessel, the body further including a conductive portion adapted to contact the liquid medium. In such case, the at least one conductor is adapted to electrically communicate with the conductive portion of the body. The at least one conductor may comprise a conductive liquid within the body in contact with the conductive portion of the body, as well as a solid conductor connecting the conductive liquid to the external structure.

The bioprocessing vessel may comprise a port for receiving the at least one sensor. The at least one sensor may be connected to a controller for monitoring one or more operating parameters of the system based on the signal from the at least one sensor. The one or more operating parameters may include any or all of liquid level, temperature, pH, oxygen, dissolved oxygen (DO), biomass/cell density, glucose, and lactate.

The system may also include a plurality of conductors for detecting a level of the liquid medium when present in the bioprocessing vessel. The plurality of conductors may comprise a plurality of pins extending into an interior compartment of the bioprocessing vessel at different heights.

The external structure may comprise a ground connection or a controller. The bioprocessing vessel may be selected form the group consisting of a bioreactor, a bioreactor having a fixed bed, a plastic bioreactor, a flexible bag, a bioreactor having a structured fixed bed, and a stirred tank bioreactor.

According to a further aspect of the disclosure, a system for culturing cells, includes a plastic bioreactor adapted to receive a liquid medium for culturing cells. At least one sensor is provided for sensing a parameter of the plastic bioreactor and producing a signal indicative thereof. At least one conductor is adapted to provide electrical communication between the liquid medium in the plastic bioreactor and a structure external to the plastic bioreactor to achieve equipotential for reducing noise in the signal from the at least one sensor.

The at least one conductor comprises a conductive pin or wire directly or indirectly connecting the liquid medium with the external structure. The at least one sensor comprises a body having a non-conductive portion connected to the plastic bioreactor, the body further including a conductive portion adapted to contact the liquid medium, the at least one conductor adapted to electrically communicate with the conductive portion of the body. The at least one conductor comprises a conductive liquid within the body in contact with the conductive body portion, as well as the at least one conductor further comprises a solid conductor connecting the conductive liquid to the external structure.

The plastic bioreactor may include a port for receiving the at least one sensor. The at least one sensor is connected to a controller for monitoring one or more operating parameters of the system based on the signal from the at least one sensor. The one or more operating parameters of the system is selected from the group consisting of liquid level, temperature, pH, oxygen, dissolved oxygen (DO), biomass/cell density, glucose, and lactate.

The system further includes a plurality of conductors for detecting a level of the liquid medium when present in the plastic bioreactor. The plurality of conductors comprise a plurality of pins extending into an interior compartment of the plastic bioreactor at different heights. The structure external to the plastic bioreactor comprises a ground connection.

The plastic bioreactor is selected from the group consisting of a plastic bioreactor having a fixed bed, a flexible plastic bag, a plastic bioreactor having a structured fixed bed, and a stirred tank plastic bioreactor.

According to a further aspect of the disclosure, an apparatus for use in connection with bioprocessing including a liquid medium and an external structure. The apparatus comprises a sensor for contacting the liquid medium, the sensor having a conductive material in electrical communication with the liquid medium. A conductor is adapted to provide electrical communication between the liquid medium via the conductive material of the sensor and the external structure to achieve equipotential.

In one embodiment, the sensor comprises a tubular body, and the conductive material comprises a portion of the tubular body. The conductor comprises a wire connected between the conductive material and the external structure. The conductive material comprises a liquid within the tubular body. The sensor may comprise a probe within the tubular body and in contact with the conductive material. The apparatus may form part of a bioprocessing vessel, which in turn may form part of a bioprocessing system.

A further aspect of the disclosure relates to a method of improving measurement of one or more process parameters by at least one sensor associated with a bioprocessing vessel formed of a material insulative to a liquid medium. The method comprises providing electrical communication between the liquid medium in the bioprocessing vessel and an external structure to achieve equipotential for reducing noise in a signal produced by the at least one sensor.

In one embodiment, the providing step comprises providing a conductor in electrical communication with the liquid medium and the external structure. The providing step may comprise providing a conductor in electrical communication with a conductive portion of the at least one sensor contacting the liquid medium and the external structure.

The method may further include the step of detecting a level of the liquid medium using a plurality of conductors extending into an interior compartment of the bioprocessing vessel at different heights. Still further, the method may include culturing cells in the bioprocessing vessel.

### BRIEF DESCRIPTION OF THE DRAWINGS

The accompanying drawings incorporated in and forming a part of the specification, illustrate several aspects of this disclosure, and together with the description serve to explain the principles of the disclosure. In the drawings:

FIG. 1 illustrates the prior art approach to utilizing sensors or probes in disposable cell culture or bioprocessing vessels;

FIG. 2 shows a representative cell culture system including an insulative (e.g., disposable/plastic) bioprocessing vessel;

FIG. 3 illustrates one embodiment of the disclosure with a sensor implementation adapted for reducing or eliminating signal noise;

FIG. 4 illustrates another embodiment of a sensor implementation adapted for reducing or eliminating signal noise;

FIG. 5 illustrates yet another embodiment of the disclosure with a sensor/probe implementation further providing level sensing capability;

FIG. 6 is a front perspective view of an embodiment of a bioprocessing vessel in the form of a bioreactor;

FIG. 6A shows a perspective view of the bioreactor of FIG. 6, with enlarged partial views of a structured bed and impeller;

FIGS. 7, 8, and 9 show alternative designs of bioprocessing vessels adapted for use with at least one sensor and potentially benefitting from the various aspects of the disclosure; and

FIG. 10 is a graph with experimental data showing pH readings from a sensor associated with an insulative (plastic, disposable) bioreactor before and after achieving equipotential using the techniques of the present disclosure.

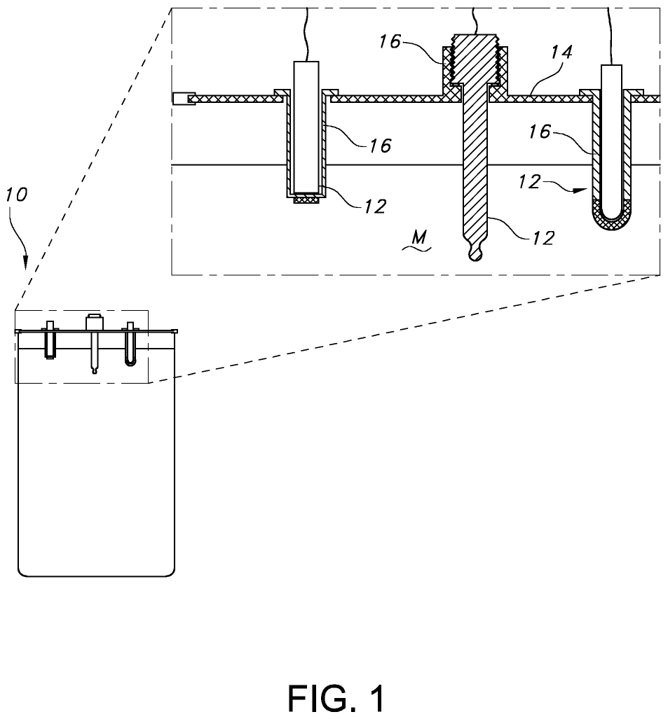

### DETAILED DESCRIPTION

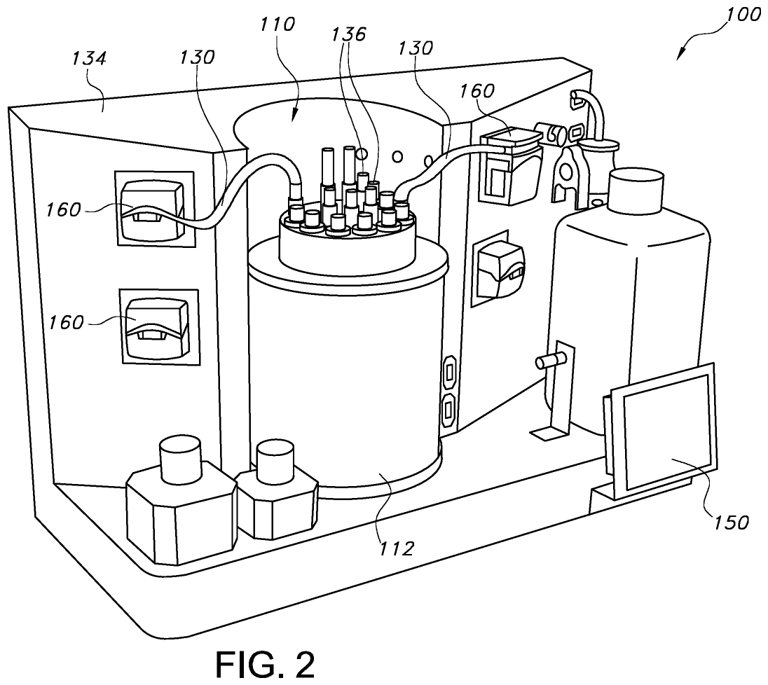

Reference is now made to FIG. 2, which illustrates an exemplary bioprocessing system 100. The integrated system 100 of this example includes a bioprocessing vessel in the form of a cell culture vessel, such as for example a bioreactor 110. Vessel 110 may also be a mixing vessel or media vessel. The bioreactor 110 includes a housing 112 made of a material insulative to the liquid therein used for culturing cells, such as plastic in the case of a disposable bioreactor (including possibly a dimensionally unstable material, such as a flexible bag or the like).

One or more sensors 136 may also be associated with the bioreactor 110. The sensor(s) may include, for example pH, dissolved oxygen, temperature, liquid level, cell density/biomass, glucose, lactate, or any sensor for measuring other desirable parameters associated with bioprocessing. The system 100 may also include tubing 130, which may be connected to a reservoir, such as a bottle , for culture media circulation.

A controller (in this example shown integrated within system docking station) 134 may form part of the system 100 for providing process control functionalities and standard parameter monitoring. The controller 134 may be associated with the one or more sensors 136 for monitoring one or more process parameters, including but not limited to pH, DO, cell density, temperature, pressure, and/or fluid levels. The controller 134 may be connected to a display 150 for displaying parameters and other aspects for control of the system 100. The controller 134 may also be associated with a pump 160, such as a peristaltic pump, for circulating fluid via the tubing 130.

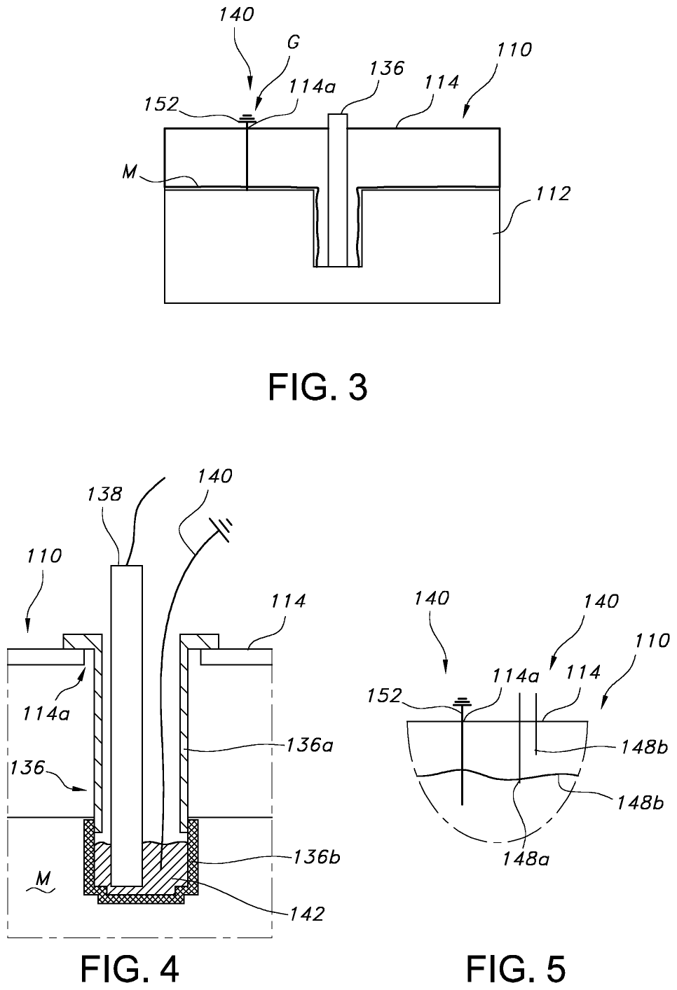

According to one aspect of the disclosure, and with reference to FIG. 3, the bioprocessing vessel housing 112 is formed of an insulative material, such as plastic, or otherwise made in a manner or of a material that serves to insulate the liquid culture medium M, thus preventing it from achieving equipotential with the sensor 136 in contact with the medium M as a result. In this particular embodiment, at least one conductor 140 shown as a conductive pin 152 (but otherwise implemented via another conductor such as a conductive wire or cable)is included with the vessel 110 for achieving equipotential for the liquid culture media M therein, so that the deleterious effects of noise on the measurement of any process parameter(s) using an associated sensor 136 are minimized, reduced or eliminated.

While an elongated structure is shown, the conductor 140 may take any shape or form, and may comprise any electrically conductive material (e.g., metals, such as stainless steel or copper for example). As illustrated, the conductor 140 may extend through a portion of the vessel 110 to connect the culture media M with an external structure, such as a connection to the ground G, or possibly using controller 134. As one example shown in FIG. 3, the pin 152 may extend through a port 114a in the lid 114, but could extend through any other portion of the vessel 110.

Turning specifically to FIG. 4, a further embodiment of a bioprocessing arrangement that may benefit from aspects of this disclosure is shown. In this version, the sensor 136 includes a tubular body 136a (also sometimes referred to as a “thermowell” in the case of a temperature sensor, which may be of the threaded, tapered, or welded variety) adapted for reducing or eliminating noise when associated with a probe portion 138 of the sensor that extends into the tubular body. This is achieved by providing such sensor 136 with a conductive portion 136b in electrical communication with the liquid media M and a conductor 140 in direct or indirect contact with the conductive portion 136b to ensure electrical communication therebetween. The remainder of the tubular body 136a including the portion for engaging the associated bioprocessing vessel, (such as a bioreactor) 110 via a port 114a in a lid 114, may be non-conductive.

The conductor 140 comprises a conductive material (such as a metal (e.g., stainless steel or copper) and may be inserted into the tubular body 136a from a position external to the bioprocessing vessel 110. For example, the conductor 140 may comprise an elongated structure, such as a solid wire or pin, having one end portion passed into the tubular body 136a and another end portion connected to an external structure (such as, for example, the ground or the controller 134). The conductor 140 may be placed in direct contact with conductive portion 136b via a screw or other mechanical fastener, or via welding, adhesive or other fastening arrangement.

The tubular body 136a may also be provided internally with a conductive material 142, such as a liquid (e.g., water or gel). This conductive material 142 serves to establish an indirect connection between the conductive portion 136b and the conductor 140 to ensure electrical communication is achieved. As can be appreciated, these arrangements serve to achieve equipotential between the sensor 136 and the liquid media in the bioprocessing vessel 110, thus potentially eliminating or significantly reducing the effects of electromagnetic noise on signals from the probe portion 138 indicative of process parameters.

When provided with a bioprocessing vessel (such as a bioreactor) 110, any conductors present may additionally be used to measure the level of liquid within the bioprocessing vessel 110 and/or detect the presence of foam. Specifically, with reference to FIG. 5, the difference of detectable electrical signal (e.g., potential, impedance, capacitance) between the conductors 140 formed by pin 152 and additional conductors 140, such as pins 148a, 148b, at different heights would not be the same when contacted by liquid or foam. This potential difference allows the measurement of several levels in combination with grounding conductor 140 shown in FIG. 3.

In such case, one skilled in the art can appreciate that the lowest conductor (148a) must remain submerged in order for this arrangement to work. As the levels of liquid or foam increase or decrease, the difference may be detected by the different levels in contact with the conductors 140 and thus provide an indication of the level of liquid or foam. Advantageously, this provides functionality to a bioreactor that both reduces the noise of the sensor probes and improves liquid level and foam detection thereby enhancing media level control without the need to use load cells. An example of such a level sensor arrangement that may be used in connection with the conductor 140 according to the disclosure is a CM-ENS liquid level relay, distributed by ABB Ltd. (e.g., three electrode version may provide the conductors 140 for liquid level sensing and grounding).

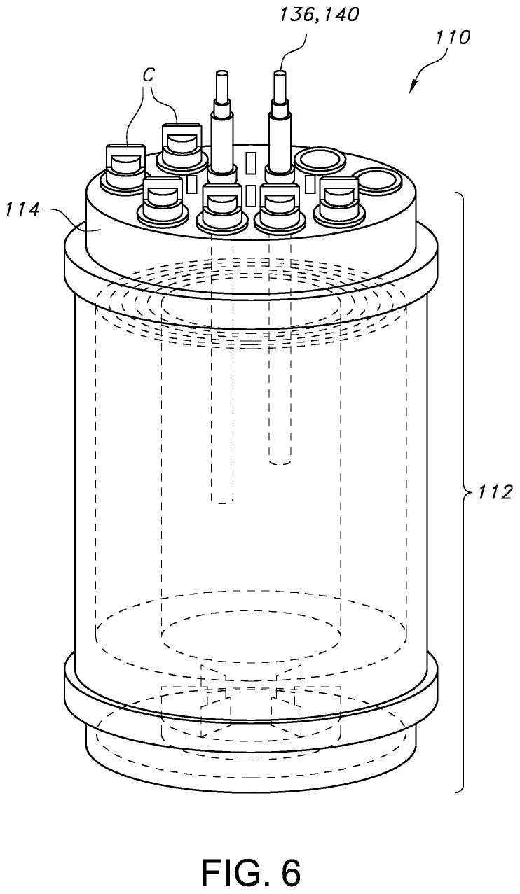

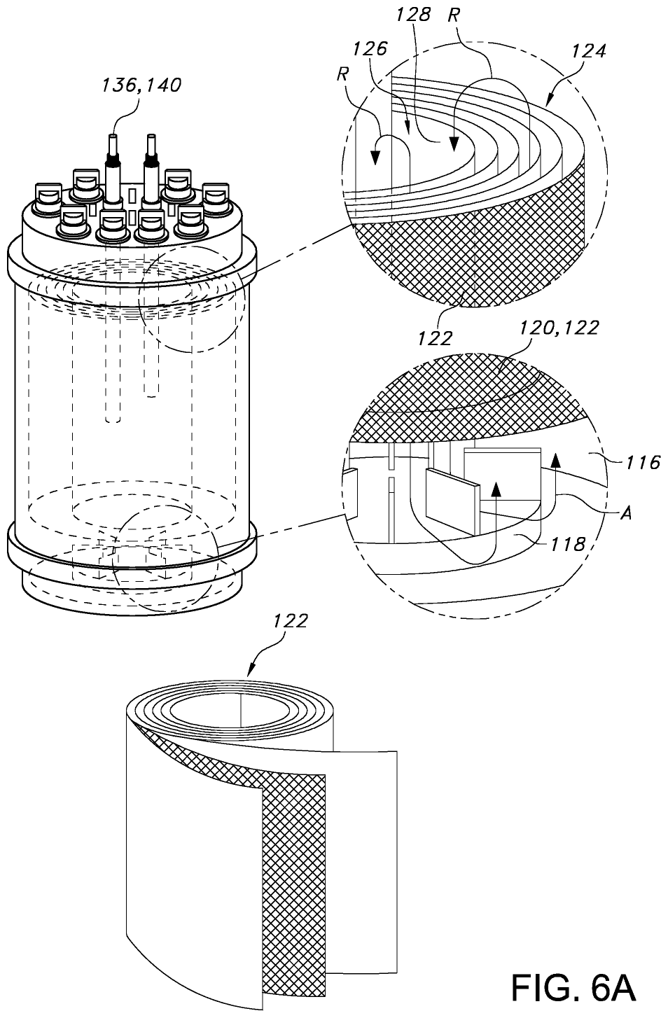

Various types of bioprocessing vessels with sensors in need of a reduction in signal noise as a result of the lack of equipotential may benefit from the foregoing teachings. As perhaps best understood with reference to FIGS. 6 and 6A, the housing 112 of a bioreactor 110 may have one or more compartments or chambers for transmitting a flow of fluid, gas, or both, as well as one or both of a sensor 136 or conductor 140 according to the above teachings. As shown, a first chamber 116 at or near a base of the bioreactor housing 112 is provided, and may include an agitator 118 for causing fluid flow. The agitator may, for example, be in the form of a magnetically driven impeller, in which case it may further contribute to the issue of electromagnetic noise. However, the agitator 118 could alternatively or additionally be in the form of an impeller with a mechanical coupling to the base, an external pump forming part of a fluid circulation system, or any other device for causing fluid circulation within the bioreactor. As a result of the agitation provided, fluid may then flow upwardly (as indicated by arrows A in FIG. 6A) into an annular chamber 120 along an outer or peripheral portion of the bioreactor housing 112.

Liquid exiting the chamber 120 passes to a headspace formed by a chamber 124 on one (upper) side of the bed 122, where the liquid is exposed to a gas (such as oxygen). Liquid may then flow radially inwardly to a central chamber 126 to return to the lower entrance leading to the chamber 124 including the bed 122. This central chamber 126 can be columnar in nature and may be formed by an imperforate conduit or tube 128 or rather formed by the central opening of the bed 122 (which is illustrated as taking the form of a structured spiral bed). The chamber 126 returns the liquid to the first chamber 116 (return arrow R) for recirculation through the bioreactor 110, such that a continuous loop results (“bottom to top” in this version, but it could be reversed).

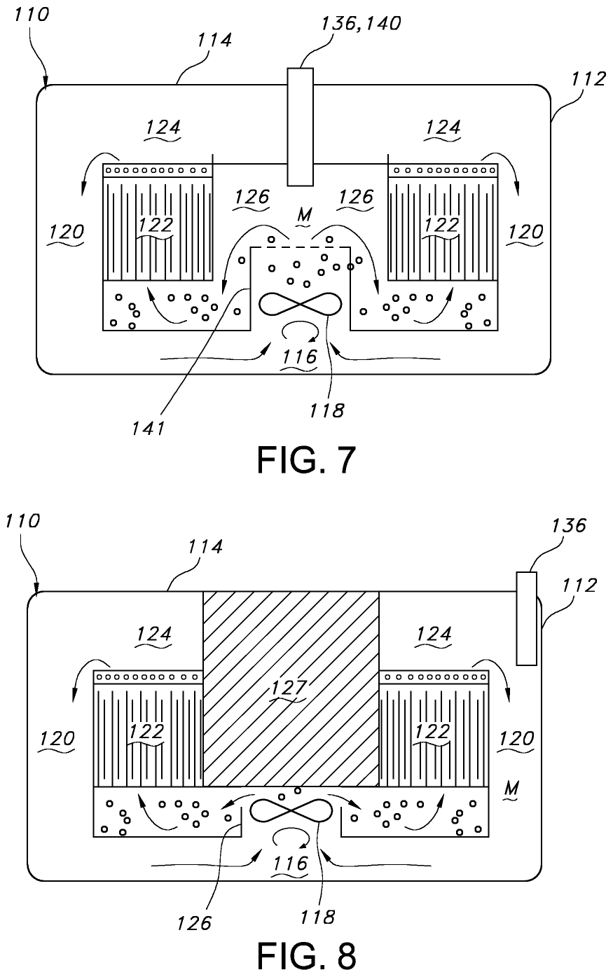

As shown in FIG. 7, an alternative embodiment of a bioreactor 110 is illustrated, which as above comprises a housing 112. In this embodiment, liquid is circulated by an agitator 118 in a housing 141 in or near the base chamber 116 and flows to a central chamber 126 first (which may be associated with the sensor 136/conductor 140), then radially outwardly to pass vertically through a structured fixed bed 122 (such as the version shown in FIGS. 6 and 6A. The liquid media upon exiting the upper portion of the bed 122 then returns along a chamber 120 radially outward of the bed 122, and is returned to the agitator 118 for repeating the cycle.

FIG. 8 illustrates an embodiment of a bioprocessing vessel similar to that of FIG. 7, with the exception that the central chamber is omitted, taking the form of a solid or closed (i.e., no path for liquid therethrough) core 127. In this embodiment, the liquid may exit a central chamber 126 for agitator 118 radially through (side) openings, and travel essentially as previously described, returning via a base chamber 116. In view of the solid or closed nature of the central chamber 126, sensing function may be achieved by introducing a sensor 136/conductor 140 into the outer chamber 120 along one or more sides of the bioreactor 110.

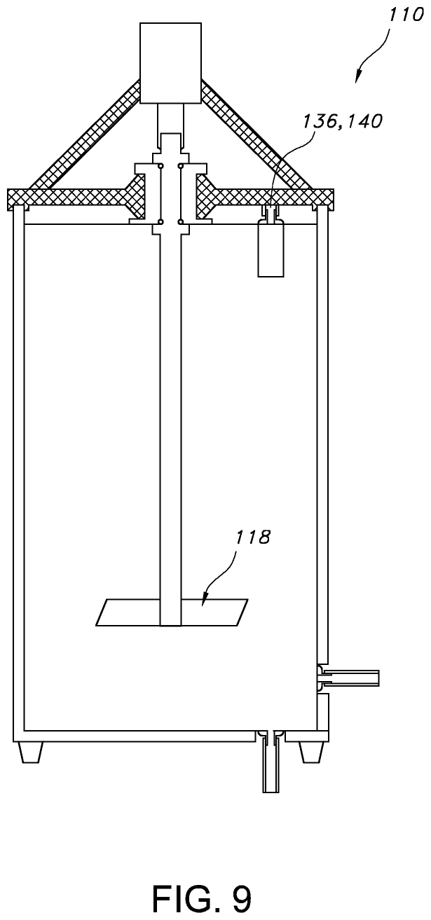

While the foregoing embodiments illustrate so-called fixed bed bioreactors, it should be appreciated that the application of the disclosed concepts is not so limited. Rather, the aspects of this disclosure may readily be applied to other forms of vessels pertaining to bioprocessing, such as for example a media or harvest vessel, a rocking bioreactor or a stirred tank bioreactor. Such a stirred tank bioreactor 110 is shown in FIG. 9, which may include an agitator 118 for agitating a culture medium with cells in suspension or forming a fluidized bed, as well as a sensor 136, conductor 140, or both.

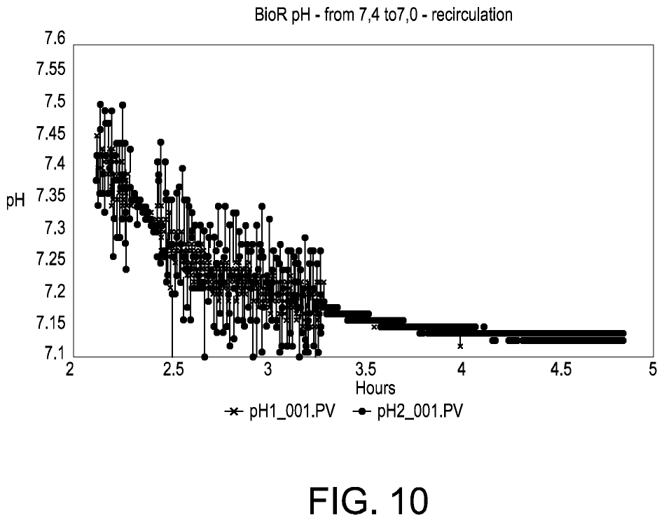

The table in FIG. 10 provides the results of experimental data of two pH probes in a bioprocessing vessel, such as a bioreactor, adapted to achieve signal noise reduction in the manner proposed herein with respect to an embodiment of the invention. At the beginning of the test, noise can be easily observed before the liquid is in equipotential. However, between 3 and 3.5 hours, the data illustrates that the liquid achieved equipotential due to adding a conductor to the bioprocessing vessel (per the embodiment in FIG. 5), thus ensuring electric conduction such that noise is greatly reduced. Summarizing, this disclosure may be considered to relate to the following items in any combination:

1. A system for bioprocessing, comprising:
a bioprocessing vessel formed of a material insulative to a liquid medium when present therein; and
at least one sensor for sensing a parameter of the bioprocessing vessel and generating a signal indicative of the parameter; and
at least one conductor adapted to provide electrical communication between the liquid medium in the bioprocessing vessel and an external structure to achieve equipotential for reducing noise in the signal produced by the at least one sensor.

2. The system of item 1, wherein the at least one conductor comprises a conductive pin or wire connecting the liquid medium with the external structure.

3. The system of item 1 or item 2, wherein the at least one sensor comprises a body connected to the insulative material of the bioprocessing vessel, the body further including a conductive portion adapted to contact the liquid medium, the at least one conductor adapted to electrically communicate with the conductive portion of the body.

4. The system of item 3, wherein the at least one conductor comprises a conductive liquid within the body in contact with the conductive portion of the body.

5. The system of item 4, wherein the at least one conductor further comprises a solid conductor connecting the conductive liquid to the external structure.

6. The system of any of items 1-5, wherein the bioprocessing vessel comprises a port for receiving the at least one sensor.

7. The system of any of items 1-6, wherein the at least one sensor is connected to a controller for monitoring one or more operating parameters of the system based on the signal from the at least one sensor.

8. The system of any of items 1-7, wherein the one or more operating parameters of the system is selected from the group consisting of liquid level, temperature, pH, oxygen, dissolved oxygen (DO), biomass/cell density, glucose, and lactate.

9. The system of any of items 1-8, further including a plurality of conductors for detecting a level of the liquid medium when present in the bioprocessing vessel.

10. The system of item 9, wherein the plurality of conductors comprise a plurality of pins extending into an interior compartment of the bioprocessing vessel at different heights.

11. The system of any of items 1-10, wherein the external structure comprises a ground connection or a controller.

12. The system of any of items 1-11, wherein the bioprocessing vessel is selected form the group consisting of a bioreactor, a bioreactor having a fixed bed, a plastic bioreactor, a flexible bag, a bioreactor having a structured fixed bed, and a stirred tank bioreactor.

13. A system for culturing cells, comprising:
a plastic bioreactor adapted to receive a liquid medium for culturing cells;
at least one sensor for sensing a parameter of the plastic bioreactor and producing a signal indicative thereof; and
at least one conductor adapted to provide electrical communication between the liquid medium in the plastic bioreactor and a structure external to the plastic bioreactor to achieve equipotential for reducing noise in the signal from the at least one sensor.

14. The system of item 13, wherein the at least one conductor comprises a conductive pin or wire connecting the liquid medium with the external structure.

15. The system of item 13 or item 14, wherein the at least one sensor comprises a body having a non-conductive portion connected to the plastic bioreactor, the body further including a conductive portion adapted to contact the liquid medium, the at least one conductor adapted to electrically communicate with the conductive portion of the body.

16. The system of item 15, wherein the at least one conductor comprises a conductive liquid within the body in contact with the conductive body portion.

17. The system of item 16, wherein the at least one conductor further comprises a solid conductor connecting the conductive liquid to the external structure.

18. The system of item 13, wherein the plastic bioreactor includes a port for receiving the at least one sensor.

19. The system of any of items 13-18, wherein the at least one sensor is connected to a controller for monitoring one or more operating parameters of the system based on the signal from the at least one sensor.

20. The system of item 19, wherein the one or more operating parameters of the system is selected from the group consisting of liquid level, temperature, pH, oxygen, dissolved oxygen (DO), biomass/cell density, glucose, and lactate.

21. The system of any of items 13-20, further including a plurality of conductors for detecting a level of the liquid medium when present in the plastic bioreactor.

22. The system of item 21, wherein the plurality of conductors comprise a plurality of pins extending into an interior compartment of the plastic bioreactor at different heights.

23. The system of any of items 13-22, wherein the structure external to the plastic bioreactor comprises a ground connection.

24. The system of any of items 13-23, wherein the plastic bioreactor is selected from the group consisting of a plastic bioreactor having a fixed bed, a flexible plastic bag, a plastic bioreactor having a structured fixed bed, and a stirred tank plastic bioreactor.

25. An apparatus for use in connection with bioprocessing including a liquid medium and an external structure, comprising:
a sensor for contacting the liquid medium, the sensor having a conductive material in electrical communication with the liquid medium; and
a conductor adapted to provide electrical communication between the liquid medium via the conductive material of the sensor and the external structure to achieve equipotential.

26. The apparatus of item 25, wherein the sensor comprises a tubular body, and the conductive material comprises a portion of the tubular body.

27. The apparatus of item 25 or item 26, wherein the conductor comprises a wire connected between the conductive material and the external structure.

28. The apparatus of item 26 or 27, wherein the conductive material further comprises a liquid within the tubular body.

29. The apparatus of any of items 26-28, wherein the sensor comprises a probe within the tubular body and in contact with the conductive material.

30. A bioprocessing vessel including the apparatus of any of items 25-29.

31. A bioprocessing system including the bioprocessing vessel of item 30.

32. A method of improving measurement of one or more process parameters by at least one sensor associated with a bioprocessing vessel formed of a material insulative to a liquid medium, comprising:
providing electrical communication between the liquid medium in the bioprocessing vessel and an external structure to achieve equipotential for reducing noise in a signal produced by the at least one sensor.

33. The method of item 32, wherein the providing step comprises providing a conductor in electrical communication with the liquid medium and the external structure.

34. The method of item 32, wherein the providing step comprises providing a conductor in electrical communication with a conductive portion of the at least one sensor contacting the liquid medium and the external structure.

35. The method of any of items 32-34, further including the step of detecting a level of the liquid medium using a plurality of conductors extending into an interior compartment of the bioprocessing vessel at different heights.

36. The method of any of items 32-35, further including the step of culturing cells in the bioprocessing vessel.
As used herein, the following terms have the following meanings:
“A”, “an”, and “the” as used herein refers to both singular and plural referents unless the context clearly dictates otherwise. By way of example, “a compartment” refers to one or more than one compartment.
“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.
“Comprise”, “comprising”, “comprises” and “comprised of” as used herein are synonymous with “include”, “including”, “includes” or “contain”, “containing”, “contains” and are inclusive or open-ended terms that specifies the presence of what follows, e.g., such does not exclude or preclude the presence of additional, non-recited components, features, element, members, steps, known in the art or disclosed therein.
While certain embodiments have been shown and described herein, it will be obvious to those skilled in the art that such embodiments are provided by way of example only. Numerous variations, changes, and substitutions will now occur to those skilled in the art without departing from the invention. For example, while the bioreactor is shown in a vertical orientation, it could be used in any orientation. It should be understood that various alternatives to the embodiments of the invention described herein may be employed in practicing the invention. For instance, sensors of all varieties, such as temperature sensors, pH sensors, dissolved oxygen sensors, biomass or cell density sensors, implemented in any kind of bioprocessing vessel where sensing is useful or necessary during the process can benefit from such disclosure. The term “vessel” is also considered to include any container, conduit, or other device associated with or used in the course of bioprocessing, and is not limited to any particular form. It is intended that the following claims define the scope of the protection under the applicable law and that methods and structures within the scope of these claims and their equivalents be covered thereby.

1. A system for bioprocessing, comprising:
a bioprocessing vessel formed of a material insulative to a liquid medium when present therein;
at least one sensor for sensing a parameter of the bioprocessing vessel and generating a signal indicative of the parameter; and
at least one conductor adapted to provide electrical communication between the liquid medium in the bioprocessing vessel and an external structure to achieve equipotential for reducing noise in the signal produced by the at least one sensor.

2. The system of claim 1, wherein the at least one conductor comprises a conductive pin or wire connecting the liquid medium with the external structure.

3. The system of claim 1, wherein the at least one sensor comprises a body connected to the insulative material of the bioprocessing vessel, the body further including a conductive portion adapted to contact the liquid medium, the at least one conductor adapted to electrically communicate with the conductive portion of the body.

4. The system of claim 3, wherein the at least one conductor comprises a conductive liquid within the body in contact with the conductive portion of the body.

5. The system of claim 4, wherein the at least one conductor further comprises a solid conductor connecting the conductive liquid to the external structure.

6. The system of claim 1, wherein the bioprocessing vessel comprises a port for receiving the at least one sensor.

7. The system of claim 1, wherein the at least one sensor is connected to a controller for monitoring one or more operating parameters of the system based on the signal from the at least one sensor.

8. The system of claim 7, wherein the one or more operating parameters of the system is selected from the group consisting of liquid level, temperature, pH, oxygen, dissolved oxygen (DO), biomass/cell density, glucose, and lactate.

9. The system of claim 1, further including a plurality of conductors for detecting a level of the liquid medium when present in the bioprocessing vessel.

10. The system of claim 9, wherein the plurality of conductors comprise a plurality of pins extending into an interior compartment of the bioprocessing vessel at different heights.

11. The system of claim 1, wherein the external structure comprises a ground connection or a controller.

12. The system of claim 1, wherein the bioprocessing vessel is selected form the group consisting of a bioreactor, a bioreactor having a fixed bed, a plastic bioreactor, a flexible bag, a bioreactor having a structured fixed bed, and a stirred tank bioreactor.

13.-24. (canceled)

25. An apparatus for use in connection with bioprocessing including a liquid medium and an external structure, comprising:
a sensor for contacting the liquid medium, the sensor having a conductive material in electrical communication with the liquid medium; and
a conductor adapted to provide electrical communication between the liquid medium via the conductive material of the sensor and the external structure to achieve equipotential.

26. The apparatus of claim 25, wherein the sensor comprises a tubular body, and the conductive material comprises a portion of the tubular body.

27. The apparatus of claim 26, wherein the conductor comprises a wire connected between the conductive material and the external structure.

28. The apparatus of claim 26, wherein the conductive material comprises a liquid within the tubular body.

29. The apparatus of claim 26, wherein the sensor comprises a probe within the tubular body and in contact with the conductive material.

30.-31. (canceled)

32. A method of improving measurement of one or more process parameters by at least one sensor associated with a bioprocessing vessel formed of a material insulative to a liquid medium, comprising:
providing electrical communication between the liquid medium in the bioprocessing vessel and an external structure to achieve equipotential for reducing noise in a signal produced by the at least one sensor.

33. The method of claim 32, wherein the providing step comprises providing a conductor in electrical communication with the liquid medium and the external structure.

34. The method of claim 32, wherein the providing step comprises providing a conductor in electrical communication with a conductive portion of the at least one sensor contacting the liquid medium and the external structure.

35. The method of any of the claim 32, further including the step of detecting a level of the liquid medium using a plurality of conductors extending into an interior compartment of the bioprocessing vessel at different heights.

36. The method of claim any of the claims 32, further including the step of culturing cells in the bioprocessing vessel.
